(19) United States
(12) Patent Application Publication
(10) Pub. No.: US 2005/0042689 A1
(43) Pub. Date: Feb. 24, 2005

(54) Diagnosing sjogren's syndrome

(76) Inventors: Ammon B. Peck, Gainesville, FL (US); Seunghee R. Cha, Gainsville, FL (US); Vijayakumar K. Ramiya, Gainesville, FL (US); Juehua Gao, Gainesville, FL (US); Debra Emily Humphreys-Baher, Grainesville, FL (US); Michael G. Humphreys-Beher, Gainesville, FL (US)

Correspondence Address: AKERMAN SENTERFITT, P.O. BOX 3188, WEST PALM BEACH, FL, 33402-3188, US

(21) Appl. No.: 10/871,137

(22) Filed: Jun. 18, 2004

Related U.S. Application Data
(60) Provisional application No. 60/479,545, filed on Jun. 18, 2003.

(51) Int. Cl. G01N 33/53; G01N 33/567; G01N 33/537; G01N 33/543

(52) U.S. Cl. 435/7.2

(57) ABSTRACT

The invention includes compositions and methods for detection of antibodies to the muscarinic adrenergic receptor type 3 (M3R), useful in the diagnosis of autoimmune diseases including Sjögren's sydrome.

This application claims the benefit of provisional U.S. application Ser. No. 60/479,545 filed Jun. 18, 2003 which is hereby incorporated by reference in its entirety.

### STATEMENT REGARDING FEDERALLY SPONSORED RESEARCH

This invention was made with U.S. government support under grant numbers R01 DE10515 and R41 AI47483-01 both awarded by the National Institutes of Health. The U.S. government may have certain rights in the invention.

### FIELD OF THE INVENTION

This invention relates generally to the fields of medicine and immunology. More particularly, the invention relates to compositions and methods for detecting anti-type-3 muscarinic acetylcholine receptor autoantibodies for diagnosing Sjögren's syndrome and other rheumatatic autoimmune diseases, such as Scleroderma.

### BACKGROUND

Sjögren's syndrome (SjS) is an autoimmune disease that manifests clinically as a loss of secretory responses in the salivary glands (leading to dry mouth or xerostomia) and lacrimal glands of the eye (leading to dry eyes or xerophthalmia). SjS occurs in two forms: primary SjS (not associated with other autoimmune diseases) and secondary SjS (associated with another autoimmune disease). As an example, secondary SjS may develop in patients suffering from rheumatoid arthritis (RA) or systemic lupus erythematosis (SLE). Present methods for diagnosing SjS are based on the combined findings of 1) dry mouth and eyes; 2) histological findings of lymphocytic infiltration into salivary glands; and 3) presence of anti-nuclear antibodies (the ANA test) in serum. While useful, the ANA test has its limits for diagnosing SjS. For example, a significant portion (about 40-50%) of SjS patients do not have detectable levels of anti-nuclear antibodies. Moreover, the ANA test is not specific for SjS as it detects other autoimmune diseases, including RA and SLE.

Thus, there is an urgent need in the art to detect and diagnose autoimmune disease, at the early stages of the disease.

### SUMMARY

The invention relates to the use of a membrane-associated (or lipid-associated) form of muscarinic acetylcholine type-3 receptor (M3R) to diagnose SjS. Autoantibodies against M3R appear to be present in the majority of SjS patients and perhaps all SjS patients if a highly sensitive test is used. Thus, membrane-associated forms of M3R can be used in immunoassays to capture and detect anti-M3R autoantibodies that might be present in a biological sample. Although other agents that bind anti-M3R autoantibodies (e.g., non-membrane associated M3R, peptide fragments of M3R or anti-idiotypic antibodies) might be used, membrane-associated forms of M3R are preferred because they have the tertiary structure that more closely resembles native forms of M3R (which are membrane-associated in situ) and anti-M3R autoantibodies are thought to recognize tertiary epitopes of M3R. Experiments have shown that assays utilizing membrane-associated forms of M3R are more sensitive than non-membrane associated forms of M3R.

Accordingly, in one aspect the invention provides a method for detecting anti-M3R antibodies in a subject. The method includes the steps of: (a) obtaining a biological sample from the subject; and (b) analyzing the sample for the presence of antibodies that specifically bind membrane-associated M3Rs. Detection of antibodies bound to M3R are preferably detected by FACS analysis, although other methods known in the art can be used. The presence of autoantibodies in the sample indicates that the subject has SjS or, to a lesser extent, scleroderma. In some versions of the method, the membrane-associated M3R is on the surface of a cell, while in other versions, it is not associated with a cell.

In a preferred embodiment, the invention provides a method for diagnosing Sjögren's syndrome in a subject, the method comprising the steps of obtaining a biological sample from the subject; analyzing the sample of the presence of an antibody that specifically binds a membrane-associated M3R; wherein the presence of the antibody in the sample indicates that the subject has Sjögren's syndrome.

In another preferred embodiment, the biological sample is a fluid selected from the group consisting of blood, blood serum, saliva, tears, mucus and ascites fluid. Preferably, the biological sample is contacted with a cell that expresses a membrane-associated molecule, such as for example, M3R.

In another preferred embodiment, a cell is contacted with a nucleic acid molecule comprising an isolated nucleic acid encoding a M3R antigen. Preferably, the cell expresses the nucleic acid molecule encoded M3R, on the cell surface. Preferably, the cell expressing the M3R molecule is membrane-associated.

In another preferred embodiment, the cell is stably transformed with the nucleic acid molecule encoding a M3R antigen. Preferably, the isolated nucleic acid encodes a human M3R antigen.

In another preferred embodiment, the cell is a CHO cell, Hep2 cell or a JURKAT cell. However, any cell that can express the M3R antigen can be used.

In another preferred embodiment, the invention provides a method for detecting anti-M3R antibodies in a biological sample, the method comprising the steps of obtaining a biological sample; and analyzing the sample of the presence of an antibody that specifically binds a membrane-associated M3R. Preferably, the biological sample is a fluid selected from the group consisting of blood, blood serum, saliva, tears, mucus and ascites fluid.

Other aspects of the invention are described infra.

### BRIEF DESCRIPTION OF THE DRAWINGS

The invention may be better understood by referring to the following drawings, in which:

FIG. 1 is an agarose gel showing a 1.7 kbp PCR product representing the M3R open reading frame DNA amplified from the human cell line, JURKAT.

FIG. 2 is an agarose gel showing a restriction enzyme digestion of plasmid DNA from transformed clones.

FIG. 3 is a series of six graphs showing results of flow cytometric analyses using control CHO cells (left panels) and transfected M3R-CHO cells (right panels) exposed to sera from two patients with SjS and a patient with an unrelated connective tissue disease. In the upper two panels on the right, the M3R-CHO cells which bound anti-M3R autoantibodies from SjS patient sera are indicated by the peaks in the window labeled M1.

### DETAILED DESCRIPTION

The invention provides methods, compositions, devices, and systems for detecting anti-M3R antibodies in a subject. The methods, compositions, devices, and systems of the invention relate to the use of a membrane-associated M3R as an agent to detect the presence of anti-M3R autoantibodies in a biological sample taken from the subject. The presence of such antibodies in the sample, in the context of other potential disease markers, is diagnostic of SjS in the subject.

Definitions

Prior to setting forth the invention, definitions of certain terms which are used in this disclosure are set forth below:

As used herein, the singular forms “a”, “an” and “the” include plural referents unless the context clearly dictates otherwise.

A “polynucleotide” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified polynucleotides such as methylated and/or capped polynucleotides.

“Recombinant,” as applied to a polynucleotide, means that the polynucleotide is the product of various combinations of cloning, restriction and/or ligation steps, and other procedures that result in a construct that is distinct from a polynucleotide found in nature.

A “gene” refers to a polynucleotide or portion of a polynucleotide comprising a sequence that encodes a protein. For most situations, it is desirable for the gene to also comprise a promoter operably linked to the coding sequence in order to effectively promote transcription. Enhancers, repressors and other regulatory sequences may also be included in order to modulate activity of the gene, as is well known in the art. (See, e.g., the references cited below).

As used herein, the term “administering a molecule to a cell” (e.g., an expression vector, nucleic acid, peptide, a delivery vehicle, agent, and the like) refers to transducing, transfecting, microinjecting, electroporating, or shooting, the cell with the molecule. In some aspects, molecules are introduced into a target cell by contacting the target cell with a delivery cell (e.g., by cell fusion or by lysing the delivery cell when it is in proximity to the target cell).

A cell has been “transformed”, “transduced”, or “transfected” by exogenous or heterologous nucleic acids when such nucleic acids have been introduced inside the cell. Transforming DNA may or may not be integrated (covalently linked) with chromosomal DNA making up the genome of the cell. In prokaryotes, yeast, and mammalian cells for example, the transforming DNA may be maintained on an episomal element, such as a plasmid. In a eukaryotic cell, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations (e.g., at least about 10).

As used herein, the term “engineered” refers to administration of a vector expressing the desired gene product into a cell, for example M3R.

As used herein, “molecule” is used generically to encompass any vector, antibody, protein, drug and the like which are used in therapy and can be detected in a patient by the methods of the invention. For example, multiple different types of nucleic acid delivery vectors encoding different types of genes which may act together to promote a therapeutic effect, or to increase the efficacy or selectivity of gene transfer and/or gene expression in a cell. The nucleic acid delivery vector may be provided as naked nucleic acids or in a delivery vehicle associated with one or more molecules for facilitating entry of a nucleic acid into a cell. Suitable delivery vehicles include, but are not limited to: liposomal formulations, polypeptides; polysaccharides; lipopolysaccharides, viral formulations (e.g., including viruses, viral particles, artificial viral envelopes and the like), cell delivery vehicles, and the like.

A “recombinant viral vector” refers to a viral vector comprising one or more heterologous genes or sequences. Since many viral vectors exhibit size-constraints associated with packaging, the heterologous genes or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying genes necessary for replication and/or encapsidation) (see, e.g., the references and illustrations below). Modified viral vectors in which a polynucleotide to be delivered is carried on the outside of the viral particle have also been described (see, e.g., Curiel, D T, et al. PNAS 88: 8850-8854, 1991).

Viral “packaging” as used herein refers to a series of intracellular events that results in the synthesis and assembly of a viral vector. Packaging typically involves the replication of the “pro-viral genome”, or a recombinant pro-vector typically referred to as a “vector plasmid” (which is a recombinant polynucleotide than can be packaged in an manner analogous to a viral genome, typically as a result of being flanked by appropriate viral “packaging sequences”), followed by encapsidation or other coating of the nucleic acid. Thus, when a suitable vector plasmid is introduced into a packaging cell line under appropriate conditions, it can be replicated and assembled into a viral particle. Viral “rep” and “cap” genes, found in many viral genomes, are genes encoding replication and encapsidation proteins, respectively. A “replication-defective” or “replication-incompetent” viral vector refers to a viral vector in which one or more functions necessary for replication and/or packaging are missing or altered, rendering the viral vector incapable of initiating viral replication following uptake by a host cell. To produce stocks of such replication-defective viral vectors, the virus or pro-viral nucleic acid can be introduced into a “packaging cell line” that has been modified to contain genes encoding the missing functions which can be supplied in trans). For example, such packaging genes can be stably integrated into a replicon of the packaging cell line or they can be introduced by transfection with a “packaging plasmid” or helper virus carrying genes encoding the missing functions.

A “detectable marker gene” is a gene that allows cells carrying the gene to be specifically detected (e.g., distinguished from cells which do not carry the marker gene). A large variety of such marker genes are known in the art. Preferred examples thereof include detectable marker genes which encode proteins appearing on cellular surfaces, thereby facilitating simplified and rapid detection and/or cellular sorting. By way of illustration, the lacZ gene encoding beta-galactosidase can be used as a detectable marker, allowing cells transduced with a vector carrying the lacZ gene to be detected by staining.

A “selectable marker gene” is a gene that allows cells carrying the gene to be specifically selected for or against, in the presence of a corresponding selective agent. By way of illustration, an antibiotic resistance gene can be used as a positive selectable marker gene that allows a host cell to be positively selected for in the presence of the corresponding antibiotic. Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e. positive/negative) markers (see, e.g., WO 92/08796, published May 29, 1992, and WO 94/28143, published Dec. 8, 1994). Such marker genes can provide an added measure of control that can be advantageous in gene therapy contexts. “Treatment” or “therapy” as used herein also refers to administering, to an individual patient, agents that are capable of eliciting a prophylactic, curative or other beneficial effect in the individual.

“Gene therapy” as used herein refers to administering, to an individual patient, vectors comprising a therapeutic gene.

The terms “polypeptide,” “peptide,” and “protein” are used interchangeably to refer to polymers of amino acids of any length. These terms also include proteins that are post-translationally modified through reactions that include glycosylation, acetylation and phosphorylation.

The terms “variant” and “amino acid sequence variant” are used interchangeably and designate polypeptides in which one or more amino acids are added and/or substituted and/or deleted and/or inserted at the N- or C-terminus or anywhere within the corresponding native sequence. In various embodiments, a “variant” polypeptide usually has at least about 75% amino acid sequence identity, or at least about 80% amino acid sequence identity, preferably at least about 85% amino acid sequence identity, even more preferably at least about 90% amino acid sequence identity, and most preferably at least about 95% amino acid sequence identity with the amino acid sequence of the corresponding native sequence polypeptide.

An “effective amount” is an amount sufficient to effect beneficial or desired clinical results. An effective amount can be administered in one or more administrations. The antibodies, peptides or vectors used as vaccines of the present invention can be administered to a patient at therapeutically effective doses to treat (including prevention) autoimmune diseases, such as for example, Sjögren's syndrome. A therapeutically effective dose refers to that amount of the compound sufficient to result in desired treatment.

As used herein, the term “fragment or segment”, as applied to a polypeptide, will ordinarily be at least about 5 contiguous amino acids, typically at least about 10 contiguous amino acids, more typically at least about 20 contiguous amino acids, usually at least about 30 contiguous amino acids, preferably at least about 40 contiguous amino acids, more preferably at least about 50 contiguous amino acids, and even more preferably at least about 60 to 80 or more contiguous amino acids in length. “Overlapping fragments” as used herein, refer to contiguous peptide fragments which begin at the amino terminal end of a protein and end at the carboxy terminal end of the protein. Each peptide fragment has at least about one contiguous amino acid position in common with the next peptide fragment, more preferably at least about three contiguous amino acid positions in common, most preferably at least about ten contiguous amino acid positions in common.

As used herein, the term “substantially pure” describes a compound (e.g., a protein or polypeptide) which has been separated from components which naturally accompany it. Typically, a compound is substantially pure when at least 10%, more preferably at least 20%, more preferably at least 50%, more preferably at least 60%, more preferably at least 75%, more preferably at least 90%, and even more preferably at least 99%, of the total material (by volume, by wet or dry weight, or by mole percent or mole fraction) in a sample is the compound of interest. Purity can be measured by any appropriate method. In the case of polypeptides, for example, purity can be measured by column chromatography, polyacrylamide gel electrophoresis, or HPLC analysis. A compound such as a protein is also substantially purified when it is essentially free of naturally associated components or when it is separated from the native contaminants which accompany it in its natural state.

A “heterologous” component refers to a component that is introduced into or produced within a different entity from that in which it is naturally located. For example, a polynucleotide derived from one organism and introduced by genetic engineering techniques into a different organism is a heterologous polynucleotide which, if expressed, can encode a heterologous polypeptide. Similarly, a promoter or enhancer that is removed from its native coding sequence and operably linked to a different coding sequence is a heterologous promoter or enhancer.

A “substantially pure nucleic acid”, as used herein, refers to a nucleic acid sequence. segment, or fragment which has been purified from the sequences which flank it in a naturally occurring state, e.g., a DNA fragment which has been removed from the sequences which are normally adjacent to the fragment such as the sequences adjacent to the fragment in a genome in which it naturally occurs. The term also applies to nucleic acids which have been substantially purified from other components which naturally accompany the nucleic acid, e.g., RNA or DNA, which has been purified from proteins which naturally accompany it in the cell.

“Homologous”, as used herein, refers to the subunit sequence similarity between two polymeric molecules, e.g., between two nucleic acid molecules such as two DNA molecules, or two polypeptide molecules. When a subunit position in both of the two molecules is occupied by the same monomeric subunit (e.g., if a position in each of two DNA molecules is occupied by adenine) then they are homologous at that position. The homology between two sequences is a direct function of the number of matching or homologous positions. For example, if 5 of 10 positions in two compound sequences are matched or homologous then the two sequences are 50% homologous, if 9 of 10 are matched or homologous, the two sequences share 90% homology. By way of example, the DNA sequences 3′ATTGCC5′ and 3′TTTCCG5′ share 50% homology.

A “promoter,” as used herein, refers to a polynucleotide sequence that controls transcription of a gene or coding sequence to which it is operably linked. A large number of promoters, including constitutive, inducible and repressible promoters, from a variety of different sources, are well known in the art and are available as or within cloned polynucleotide sequences (from, e.g., depositories such as the ATCC as well as other commercial or individual sources).

An “enhancer,” as used herein, refers to a polynucleotide sequence that enhances transcription of a gene or coding sequence to which it is operably linked. A large number of enhancers, from a variety of different sources are well known in the art and available as or within cloned polynucleotide sequences (from, e.g., depositories such as the ATCC as well as other commercial or individual sources). A number of polynucleotides comprising promoter sequences (such as the commonly-used CMV promoter) also comprise enhancer sequences. “Operably linked” refers to a juxtaposition, wherein the components so described are in a relationship permitting them to function in their intended manner. A promoter is operably linked to a coding sequence if the promoter controls transcription of the coding sequence. Although an operably linked promoter is generally located upstream of the coding sequence, it is not necessarily contiguous with it. An enhancer is operably linked to a coding sequence if the enhancer increases transcription of the coding sequence Operably linked enhancers can be located upstream, within or downstream of coding sequences. A polyadenylation sequence is operably linked to a coding sequence if it is located at the downstream end of the coding sequence such that transcription proceeds through the coding sequence into the polyadenylation sequence.

A “replicon” refers to a polynucleotide comprising an origin of replication which allows for replication of the polynucleotide in an appropriate host cell. Examples include replicons of a target cell into which a heterologous nucleic acid might be integrated (e.g., nuclear and mitochondrial chromosomes), as well as extrachromosomal replicons (such as replicating plasmids and episomes).

As used herein, the term “antibody” refers to a polypeptide or group of polypeptides which are comprised of at least one binding domain, where an antibody binding domain is formed from the folding of variable domains of an antibody molecule to form three-dimensional binding spaces with an internal surface shape and charge distribution complementary to the features of an antigenic determinant of an antigen, which allows an immunological reaction with the antigen. Antibodies include recombinant proteins comprising the binding domains, as wells as fragments, including Fab, Fab′, F(ab)2, and F(ab′) 2 fragments.

The term “polyclonal” refers to antibodies that are heterogeneous populations of antibody molecules derived from the sera of animals immunized with an antigen or an antigenic functional derivative thereof. For the production of polyclonal antibodies, various host animals may be immunized by injection with the antigen. Various adjuvants may be used to increase the immunological response, depending on the host species.

“Monoclonal antibodies” are substantially homogenous populations of antibodies to a particular antigen. They may be obtained by any technique which provides for the production of antibody molecules by continuous cell lines in culture. Monoclonal antibodies may be obtained by methods known to those skilled in the art. See, for example, Kohler, et al., Nature 256:495-497, 1975, and U.S. Pat. No. 4,376,110.

As used herein, an “antigenic determinant” is the portion of an antigen molecule that determines the specificity of the antigen-antibody reaction. An “epitope” refers to an antigenic determinant of a polypeptide. An epitope can comprise as few as 3 amino acids in a spatial conformation which is unique to the epitope. Generally an epitope consists of at least 6 such amino acids, and more usually at least 8-10 such amino acids. Methods for determining the amino acids which make up an epitope include x-ray crystallography, 2-dimensional nuclear magnetic resonance, and epitope mapping e.g. the Pepscan method described by H. Mario Geysen et al. 1984. Proc. Natl. Acad. Sci. U.S.A. 81:3998-4002; PCT Publication No. WO 84/03564; and PCT Publication No. WO 84/03506.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to marker “X” from specific species such as rat, mouse, or human can be selected to obtain only those polyclonal antibodies that are specifically immunoreactive with marker “X” and not with other proteins, except for polymorphic variants and alleles of marker “X”. This selection may be achieved by subtracting out antibodies that cross-react with marker “X” molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

Immunoassay” is an assay that uses an antibody to specifically bind an antigen (e.g., a marker). The immunoassay is characterized by the use of specific binding properties of a particular antibody to isolate, target, and/or quantify the antigen.

As used herein, the term “humanized” antibody refers to a molecule that has its CDRs (complementarily determining regions) derived from a non-human species immunoglobulin and the remainder of the antibody molecule derived mainly from a human immunoglobulin. The term “antibody” as used herein, unless indicated otherwise, is used broadly to refer to both antibody molecules and a variety of antibody derived molecules. Such antibody derived molecules comprise at least one variable region (either a heavy chain of light chain variable region) and include molecules such as Fab. fragments, Fab′ fragments, F(ab′)2 fragments, Fd fragments, Fab′ fragments, Fd fragments, Fabc fragments, Sc antibodies (single chain antibodies), diabodies, individual antibody light chains, individual antibody heavy chains, chimeric fusions between antibody chains and other molecules, and the like.

The term “variable region” as used herein in reference to immunoglobulin molecules has the ordinary meaning given to the term by the person of ordinary skill in the act of immunology. Both antibody heavy chains and antibody light chains may be divided into a “variable region” and a “constant region”. The point of division between a variable region and a heavy region may readily be determined by the person of ordinary skill in the art by reference to standard texts describing antibody structure, e.g., Kabat et al “Sequences of Proteins of Immunological Interest: 5th Edition” U.S. Department of Health and Human Services, U.S. Government Printing Office (1991).

A “therapeutic polynucleotide” or “therapeutic gene” refers to a nucleotide sequence that is capable, when transferred to an individual, of eliciting a prophylactic, curative or other beneficial effect in the individual.

The term “autoimmune inhibitor” is used to refer to a “compound” or “compounds,” including one or more molecules, antigens, and/or antibodies (alone or in combination), which when administered in an effective amount to a patient, binds to, neutralizes or inhibits circulating pathological agents and/or those on the surface of target cells, and which when placed in extracorporeal contact with the patient's body fluids effects the removal, neutralization or inhibition of complex pathological agents (including hyperproduced cytokines and autoantibodies). The autoimmune inhibitor may also comprise antibodies to a receptor of the autoantigen. A “receptor” is a protein found on the surface of a target cell or in its cytoplasm, that has a binding site with high affinity to a particular signaling substance (e.g., a cytokine, hormone, neurotransmitter, etc.). By competitively inhibiting the availability of the receptor with an analog or antibody to the receptor, the immune response to the autoimmunogen is modified or neutralized.

The term “autoimmune disease” refers to those disease states and conditions wherein the immune response of the patient is directed against the patient's own constituents, resulting in an undesirable and often terribly debilitating condition. As used herein, “autoimmune disease” is intended to further include autoimmune conditions, syndromes and the like. An “autoantigen” is a patient's self-produced constituent, which is perceived to be foreign or undesirable, thus triggering an autoimmune response in the patient, which may in turn lead to a chain of events, including the synthesis of other autoantigens or autoantibodies. An “autoantibody” is an antibody produced by an autoimmune patient to one or more of his own constituents which are perceived to be antigenic. For example, in AIDS disease the patient eventually produces autoantibodies to CD4 cells, in SLE autoantibodies are produced to DNA, while in many other types of AD autoantibodies are produced to target cells.

Patients suffering from autoimmune diseases including, e.g., rheumatoid arthritis, insulindependent diabetes mellitus, hemolytic anemias, rheumatic fever, thyroiditis, Crohn's disease, myasthenia gravis, glomerulonephritis, autoimmune hepatitis, multiple sclerosis, systemic lupus erythematosus and others, are in need of treatment in accordance with the present invention. Treatment of patients suffering from these diseases by administration of autoimmune inhibitor and/or removal of compound(s) by extracorporeal immunosorption in accordance with the present invention will alleviate the clinical manifestations of the disease and/or minimize or prevent further deterioration or worsening of the patient's condition. Treatment of a patient at an early stage of an autoimmune disease including, e.g., rheumatoid arthritis, insulin-dependent diabetes mellitus, multiple sclerosis, myasthenia gravis, systemic lupus erythematosus, or others, will minimize or eliminate deterioration of the disease state into a more serious condition.

The term “fluid” refers to blood, plasma, plasma containing leukocytes, serum, serum and leukocytes, peritoneal fluid, cerebrospinal fluid, synovial fluid, amniotic fluid, or the like, drawn from the patient in the practice of the present invention.

The phrase “differentially present” refers to differences in the quantity and/or the frequency of a marker present in a sample taken from patients having for example, M3R autoantibodies as compared to a control subject. For example, a marker can be a polypeptide which is present at an elevated level or at a decreased level in samples of patients with neural injury compared to samples of control subjects. Alternatively, a marker can be a polypeptide which is detected at a higher frequency or at a lower frequency in samples of patients compared to samples of control subjects. A marker can be differentially present in terms of quantity, frequency or both.

A polypeptide is differentially present between the two samples if the amount of the polypeptide in one sample is statistically significantly different from the amount of the polypeptide in the other sample. For example, a polypeptide is differentially present between the two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a polypeptide is differentially present between the two sets of samples if the frequency of detecting the polypeptide in samples of patients' suffering from autoimmune disorder, is statistically significantly higher or lower than in the control samples. For example, a polypeptide is differentially present between the two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

“Diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

A “test amount” of a marker refers to an amount of a marker present in a sample being tested. A test amount can be either in absolute amount (e.g., μg/ml) or a relative amount (e.g., relative intensity of signals).

A “diagnostic amount” of a marker refers to an amount of a marker in a subject's sample that is consistent with a diagnosis of autoimmune disorder. A diagnostic amount can be either in absolute amount (e.g., μg/ml) or a relative amount (e.g., relative intensity of signals).

A “control amount” of a marker can be any amount or a range of amount which is to be compared against a test amount of a marker. For example, a control amount of a marker can be the amount of a marker in a person without autoimmune disorder. A control amount can be either in absolute amount (e.g., μg/ml) or a relative amount (e.g., relative intensity of signals).

The practice of the present invention can suitably employ, unless otherwise indicated, conventional techniques of molecular biology and the like, which are within the skill of the art. Such techniques are explained fully in the literature. See e.g., Molecular Cloning: A Laboratory Manual, (J. Sambrook et al., Cold Spring Harbor Laboratory, Cold Spring Harbor, N.Y., 1989); Current Protocols in Molecular Biology (F. Ausubel et al. eds., 1987 and updated); Essential Molecular Biology (T. Brown ed., IRL Press 1991); Gene Expression Technology (Goeddel ed., Academic Press 1991); Methods for Cloning and Analysis of Eukaryotic Genes (A. Bothwell et al. eds., Bartlett Publ. 1990); Gene Transfer and Expression (M. Kriegler, Stockton Press 1990); Recombinant DNA Methodology (R. Wu et al. eds., Academic Press 1989); PCR: A Practical Approach (M. McPherson et al., IRL Press at Oxford University Press 1991); Cell Culture for Biochemists (R. Adams ed., Elsevier Science Publishers 1990); Gene Transfer Vectors for Mammalian Cells (J. Miller & M. Calos eds., 1987); Mammalian Cell Biotechnology (M. Butler ed., 1991); Animal Cell Culture (J. Pollard et al. eds., Humana Press 1990); Culture of Animal Cells, 2nd Ed. (R. Freshney et al. eds., Alan R. Liss 1987); Flow Cytometry and Sorting (M. Melamed et al. eds., Wiley-Liss 1990); the series Methods in Enzymology (Academic Press, Inc.); Techniques in Immunocytochemistry, (G. Bullock & P. Petrusz eds., Academic Press 1982, 1983, 1985, 1989); Handbook of Experimental Immunology, (D. Weir & C. Blackwell, eds.); Cellular and Molecular Immunology (A. Abbas et al., W. B. Saunders Co. 1991, 1994); Current Protocols in Immunology (J. Coligan et al. eds. 1991); the series Annual Review of Immunology; the series Advances in Immunology; Oligonucleotide Synthesis (M. Gait ed., 1984); and Animal Cell Culture (R. Freshney ed., IRL Press 1987).

Biological Methods

Methods involving conventional immunological and molecular biological techniques are described herein. Immunological methods (for example, assays for detection and localization of antigen-antibody complexes, immunoprecipitation, immunoblotting, and the like) are generally known in the art and described in methodology treatises such as Current Protocols in Immunology, Coligan et al., ed., John Wiley & Sons, New York, 1992. Techniques of molecular biology are described in detail in treatises such as Molecular Cloning: A Laboratory Manual, 2nd ed., vol. 1-3, Sambrook et al., ed., Cold Spring Harbor Laboratory Press, Cold Spring Harbor, N.Y., 2001; and Current Protocols in Molecular Biology, Ausubel et al., ed., Greene Publishing and Wiley-Interscience, New York, 1992 (with periodic updates). Cell culture techniques are generally known in the art and are described in detail in methodology treatises such as Culture of Animal Cells: A Manual of Basic Technique, 4th edition, by R Ian Freshney, Wiley-Liss, Hoboken, N.J., 2000; and General Techniques of Cell Culture, by Maureen A Harrison and Ian F Rae, Cambridge University Press, Cambridge, UK, 1994. Methods of protein purification are discussed in Guide to Protein Purification: Methods in Enzymology, Vol. 182, Deutscher M P, ed., Academic Press, San Diego, Calif., 1990.

Detecting Anti-M3R Autoantibodies in a Biological Sample

In a preferred embodiment, a method for monitoring an autoimmune disorder in a subject mammal comprises determining the amount of M3R protein present in a sample from the subject mammal being treated for or suspected of exhibiting the autoimmune disorder, wherein the sample is obtained from a tissue affected by the disorder. Such an embodiment can further comprise determining the ratio of M3R protein, variants or fragments thereof, present in an normal individual and an individual suffering from or susceptible to an autoimmune disorder, such as for example, Sjögren's syndrome.

The methods for monitoring an autoimmune disorder in a subject mammal can further comprise assaying the sample for evidence of leukocyte infiltration or tissue damage (cell injury) using standard techniques. For example, histological techniques well known to those of skill in the art can be utilized. Alternatively, standard techniques can be utilized to assay (e.g., in serum) for the presence of autoimmune antibodies associated with the particular autoimmune disorder of interest. There are internationally used diagnostic criteria for evaluation of graft rejection, with features specific for each organ. The immunohistologic evaluation of such tissues (for example, salivary glands) i.e., use of unlabeled-antibody techniques to localize and quantitate gene expression, can be enhanced by localization of M3R proteins, peptides, variants or fragments thereof, or detection of corresponding mRNAs by in situ hybridization.

Such methods for monitoring an autoimmune disorder in a subject mammal can further comprise comparing the amount or ratio determined to that present in a control sample, for example, a corresponding tissue not affected by the disorder or a subject blood sample. In instances wherein the amount of M3R mRNA or protein in the sample is greater than, that of the control sample, such a result indicates that the subject mammal exhibits or continues to exhibit the disorder. In instances wherein the amount of M3R mRNA or protein in the sample is less than, or the amount of M3R mRNA or protein in the sample is equal to than that of the control sample, such a result indicates that the subject mammal does not exhibit the disorder or that treatment for the disorder is effective. In instances wherein the ratio of M3R (diseased individual): M3R (normal individual) in the sample is greater than or equal to that in the control sample, such a result indicates that the subject mammal exhibits or continues to exhibit the disorder. Thus, M3R autoantibodies that are differentially present in an individual is diagnostic of Sjögren's syndrome.

In a preferred embodiment, the invention provides antibodies for use as diagnostic agents which detect autoimmune disorders, especially, for example, Sjögren's syndrome. In one embodiment, any of the above-described molecules can be labeled, either detectably, as with a radioisotope, a paramagnetic atom, a fluorescent moiety, an enzyme, etc. in order to facilitate its detection in, for example, in situ or in vivo assays. The molecules may be labeled with reagents such as biotin, in order to, for example, facilitate their recovery, and/or detection.

In a preferred embodiment, the invention provides a method of detecting anti-M3R antibodies in a patient suffering from or susceptible to an autoimmune disease such as Sjögren's syndrome. Preferably, the method of detecting anti-M3R antibodies of the invention includes the step of analyzing a biological sample for the presence of an antibody that specifically binds a M3R. Any suitable biological sample might be used in the method. For example, a biological sample that would normally be expected to contain immunoglobulins might be used. Typically, the sample would take the form of a bodily fluid, such as blood (and fractions thereof, such as serum or plasma), saliva, tears, mucus, and the like. Because immunoassays are known to generally work well with blood or blood fractions, these are preferred. Biological samples can be collected from a subject by any suitable method. For example, a blood sample can be collected using conventional phlebotomy procedures; a saliva sample can be collected by spitting or merely by placing a stick in the mouth and is the preferred patient sample. Blood samples can be used for verification of detection of autoantibodies.

A subject from which a biological sample can be obtained for analysis according to the invention is an animal such as a mammal, e.g., a dog, cat, horse, cow, pig, sheep, goat, primate, rat, or mouse. A preferred subject is a human being, particularly a patient suspected of having or at risk for developing an autoimmune disorder such as SjS (e.g., an individual suffering from dry eye and/or dry mouth), or a patient with a connective tissue disease (e.g., an individual diagnosed with SLE, rheumatoid arthritis, or scleroderma).

In a preferred embodiment, analysis of a biological sample for the presence of an antibody that specifically binds a M3R, an agent to which an anti-M3R antibody specifically binds is utilized. Such agents might include a native (i.e., naturally occurring) M3R or fragments, mutants or variants thereof; or a non-M3R molecule such as an anti-idiotypic antibody. A number of different native M3Rs have been characterized (e.g., amino acid sequenced), including those from human, mouse, rat, pig, orangutan, chimpanzee, cow, gorilla, guinea pig, and chicken. Generally, those M3Rs from the same species as the biological sample are preferred for particular variations of the method of the invention. Nonetheless, due to cross-reactivity, non-species matched assays may also be used. For example, rat M3R can be used to detect human anti-M3R antibodies. Because native M3Rs are membrane-associated, in order to most closely mimic their native structure, those that are in membrane-associated (or lipid-associated) form are preferred over those that are not membrane-associated (or lipid-associated).

In another preferred embodiment, a sample is contacted with a cell that expresses a membrane-associated M3R, e.g., a JURKAT, Hep2 or CHO cell engineered to express a membrane-associated M3R. In others, a biological sample is contacted with a lipid-associated M3R that is not associated with a cell (e.g., one associated covalently or non-covalently a fatty acid, a phospholipid, a micelle, a liposome, or lipid-coated substrate).

To analyze a biological sample for the presence of an anti-M3R antibody, anti-idiotypic anti-M3R or M3R proteins, peptides, variants or fragments thereof, a molecule that specifically binds to these proteins, a number of different methods might be used. In general, the sample, or a purified portion thereof, is contacted with the agent under conditions that allow agent-antibody binding. The presence of the formed agent-antibody complex is then detected as an indication that the sample contains an anti-M3R antibody. Methods for detecting antigen (the agent is the antigen in this case)-antibody complexes are well known in the art of immunology, and include techniques that utilize M3R-expressing cells as well as non-cellular methods.

Cell-based detection methods include techniques such as immunohistochemistry, immunofluorescence microscopy, or flow cytometric analysis. As an example, a cell (e.g., a JURKAT or a CHO cell) that expresses M3R on its surface is first mixed with a biological sample under conditions that allow any anti-M3R antibodies present in the sample to bind to the cell-associated M3R. After washing away unbound antibodies, the cell is then contacted with detectably labeled secondary antibody (e.g., an anti-human immunoglobulin antibody if the biological sample was derived from a human subject). The presence of detectable label (e.g., an enzyme, fluorophore, or radioisotope) on the cell after washing indicates that the sample contained anti-M3R antibodies. Suitable negative control cells include those that do not express M3R.

Non-cell based assays include immunosorbent assays (e.g., ELISA and RIA) and immunoprecipitation assays. As one example, membrane-associated M3R is immobilized on a substrate, a human serum sample is placed on the substrate under conditions that would allow binding of anti-M3R antibodies to the immobilized M3R. After washing, detectably labeled secondary antibody (e.g., an anti-human immunoglobulin antibody if the biological sample was derived from a human subject) is added to the substrate. The presence of detectable label (e.g., an enzyme, fluorophore, or radioisotope) on the substrate after washing indicates that the sample contained anti-M3R antibodies. As another example, antibodies contained within a biological sample are immobilized on a substrate, a detectably labeled M3R is then placed on the substrate under conditions that would allow binding of the immobilized anti-M3R antibodies to the M3R. The presence of detectable label remaining on the substrate after washing indicates that the sample contained anti-M3R antibodies.

For use in the invention, membrane-associated (or lipid-associated) M3R can be obtained or made by conventional methods. As an example, plasma membranes from cells expressing M3R on their cell surfaces can be isolated by known cell fractionation techniques. Alternatively, M3R can be covalently conjugated to a fatty acid or phospholipid, or incorporated into liposomes or micelles, according to known techniques.

Diagnosing SjS

Utilizing the foregoing methods and compositions, the present invention provides a method for diagnosing SjS in a subject.

In a preferred embodiment, the method includes the steps of: (a) obtaining a biological sample from the subject; and (b) analyzing the sample for the presence of an antibody that specifically binds a membrane-associated M3R, wherein the presence of antibody in the sample indicates that the subject is likely to have SjS.

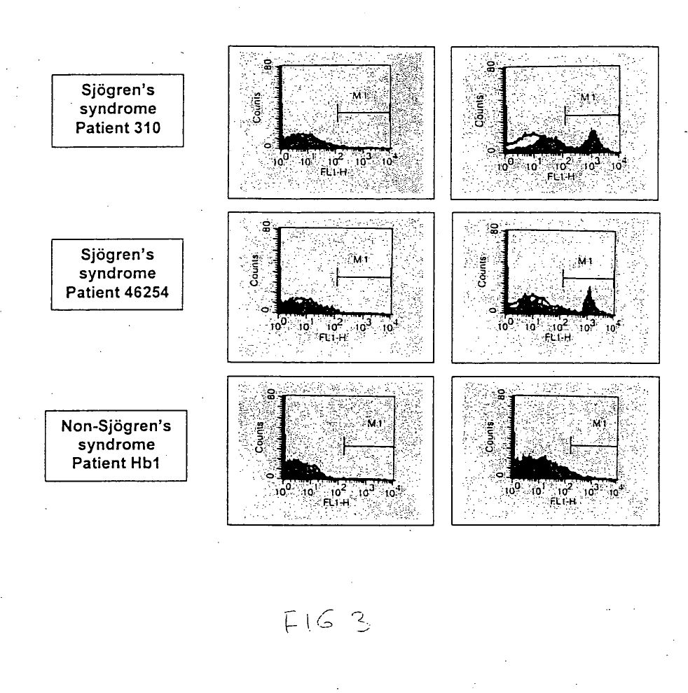

A preferred embodiment for detecting M3R is exemplified by Example 3. Briefly, M3R-transfected (M3R-Flp-In CHO) and control (non-transfected Flp-In CHO) cells were prepared and used for flow cytometric analysis as described in Example 1, using sera from normal subjects and subjects with autoimmune diseases. FIG. 3 shows typical results from control and transfected cells which were incubated with the sera from subjects with SjS, and from subjects with a connective tissue disease unrelated to SjS. Panels on the left show the graphic readout of the flow cytometer for control cells incubated with sera from the indicated subjects; panels on the right show the readouts for M3R-CHO cells incubated with the corresponding sera. The bar labeled M1 corresponds to the window indicating presence of cells with bound FITC label, i.e., those cells having bound antigen-antibody complexes secondarily labeled with FITC-labeled IgG. No M1 peak was seen with control (non-transfected) cells incubated with any of the tested sera (left panels). In contrast, a sharp M1 peak was observed following incubation of the M3R-CHO cells with sera from the two SjS patients, but not with serum from a non-SjS autoimmune patient (right panels). Similarly, no M1 peak was observed following incubation of the control and M3R-CHO cells with sera from normal subjects.

Standard techniques can also be utilized for determining the amount of the protein or proteins of interest (that is, M3R proteins, peptides, variants or fragments thereof) present in a sample. It is to be understood, that such a determination of the amount of a protein present includes determining the total amount of a protein present, and also includes, especially with respect to determining the amount of M3R protein present, determining the amount of a phosphorylated form of the protein present.

For example, standard techniques can be employed using, e.g., immunoassays such as, for example, Western blot, immunoprecipitation followed by sodium dodecyl sulfate polyacrylamide gel electrophoresis (SDS-PAGE), immunocytochemistry, and the like to determine the amount of the protein or proteins of interest present in a sample. A preferred agent for detecting a protein of interest is an antibody capable of binding to a protein of interest, preferably an antibody with a detectable label.

With respect to determining the amount of a phosphorylated form of a protein of interest that is present in a sample, such a determination can also be performed using standard techniques well known to those of skill in the art. For example, such a determination can include, first, immunoprecipitation with an antibody that is specific for a phosphorylated amino acid residue, e.g., an anti-phosphotyrosine antibody, such that all exhibiting such a phosphorylated residue in a sample will be immunoprecipitated. Second, the immunoprecipitated proteins can be contacted with a second antibody that is specific for the particular protein of interest, e.g., M3R. Alternatively, a phosphorylated protein of interest can be identified and quantitated using an antibody specific for the phosphorylated form of the particular protein itself, e.g., an antibody specific for phosphorylated M3R that does not recognize non-phosphorylated M3R.

For such detection methods, protein from the sample to be analyzed can easily be isolated using techniques which are well known to those of skill in the art. Protein isolation methods can, for example, be such as those described in Harlow and Lane (Harlow, E. and Lane, D., 1988, “Antibodies: A Laboratory Manual”, Cold Spring Harbor Laboratory Press, Cold Spring Harbor, N.Y.).

Preferred methods for the detection of the protein or proteins of interest involve their detection via interaction with a protein-specific antibody. For example, antibodies directed to a protein of interest can be utilized as described herein. Antibodies directed against M3R are described in detail in the Examples which follow. Alternatively, such antibodies can be generated utilizing standard techniques well known to those of skill in the art. Briefly, such antibodies can be polyclonal, or more preferably, monoclonal. An intact antibody, or a fragment thereof (e.g., Fab or F(ab′)₂) can, for example, be used.

In accordance with the invention, M3R antibodies, or fragments of antibodies, specific for a protein of interest can be used to quantitatively or qualitatively detect the presence of the protein. This can be accomplished, for example, by immunofluorescence techniques as described in detail in the Examples which follow. Antibodies (or fragments thereof) can, additionally, be employed histologically, as in immunofluorescence or immunoelectron microscopy, for in situ detection of a protein of interest. In situ detection can be accomplished by removing a histological specimen (e.g., a biopsy specimen) from a patient, and applying thereto a labeled antibody thereto that is directed to an M3R protein, peptide, variant or fragments thereof. The antibody (or fragment) is preferably applied by overlaying the labeled antibody (or fragment) onto a biological sample. Through the use of such a procedure, it is possible to determine not only the presence of the protein of interest, but also its distribution, its presence in lymphocytes within the sample. A wide variety of well-known histological methods (such as staining procedures) can be utilized in order to achieve such in situ detection.

Immunoassays for a protein of interest typically comprise incubating a biological sample, e.g., a biopsy or subject blood sample, of a detectably labeled antibody capable of identifying a protein of interest, and detecting the bound antibody by any of a number of techniques well-known in the art. As discussed in more detail, below, the term “labeled” can refer to direct labeling of the antibody via, e.g., coupling (i.e., physically linking) a detectable substance to the antibody, and can also refer to indirect labeling of the antibody by reactivity with another reagent that is directly labeled. Examples of indirect labeling include detection of a primary antibody using a fluorescently labeled secondary antibody.

The biological sample can be brought in contact with and immobilized onto a solid phase support or carrier such as nitrocellulose, or other solid support which is capable of immobilizing cells, cell particles or soluble proteins. The support can then be washed with suitable buffers followed by treatment with the detectably labeled fingerprint gene-specific antibody. The solid phase support can then be washed with the buffer a second time to remove unbound antibody. The amount of bound label on solid support can then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material can have virtually any possible structural configuration so long as the coupled molecule is capable of binding to an antigen or antibody. Thus, the support configuration can be spherical, as in a bead, or cylindrical, as in the inside surface of a test tube, or the external surface of a rod. Alternatively, the surface can be flat such as a sheet, test strip, etc. Preferred supports include polystyrene beads. Those skilled in the art will know many other suitable carriers for binding antibody or antigen, or will be able to ascertain the same by use of routine experimentation.

In another preferred embodiment, M3R autoantibodies can be detectably labeled by linking the same to an enzyme and use in an enzyme immunoassay (EIA) (Voller, A., “The Enzyme Linked Immunosorbent Assay (ELISA)”, 1978, Diagnostic Horizons 2:1-7, Microbiological Associates Quarterly Publication, Walkersville, Md.); Voller, A. et al., 1978, J. Clin. Pathol. 31:507-520; Butler, J. E., 1981, Meth. Enzymol. 73:482-523; Maggio, E. (ed.), 1980, ENZYME IMMUNOASSAY, CRC Press, Boca Raton, Fla.; Ishikawa, E. et al., (eds.), 1981, ENZYME IMMUNOASSAY, Kgaku Shoin, Tokyo). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by calorimetric methods which employ a chromogenic substrate for the enzyme. Detection can also be accomplished by visual comparison of the extent of enzymatic reaction of a substrate in comparison with similarly prepared standards.

Detection can also be accomplished using any of a variety of other immunoassays. For example, by radioactively labeling the antibodies or antibody fragments, it is possible to detect a protein of interest through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope (e.g. ¹²⁵I, ¹³¹I, ³⁵S or ³H) can be detected by such means as the use of a gamma counter or a scintillation counter or by autoradiography.

It is also possible to label the antibody with a fluorescent compound. When the fluorescently labeled antibody is exposed to light of the proper wavelength, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound can be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

To evaluate the antibody or antibodies, conditions for incubating the antibody or antibodies with a test sample vary. Incubating conditions depend on the format employed in the assay, the detection methods employed, the nature of the test sample, and the type and nature of the antibody used in the assay. One skilled in the art will recognize that any one of the commonly available immunological assay formats (such as, radioimmunoassays, enzyme-linked immunosorbent assays, diffusion based Ouchterlony, or rocket immunofluorescent assays, or the like) can readily be adapted to employ the antibodies of the present invention.

Auto-M3R Antibodies for Therapy

Autoimmune antibodies or inhibitor(s) of the present invention include polypeptides comprising the epitope of the antibody or biologically active fragment thereof, or polypeptide that is functional in conferring protection in the individual suffering from autoimmune disease, or functionally conserved fragments or amino acid variants thereof. Identification of the epitope is a matter of routine experimentation. Most typically, one would conduct systematic substitutional mutagenesis of the compound molecule while observing for reductions or elimination of cytoprotective or neutralizing activity. In any case, it will be appreciated that due to the size of many of the antibodies, most substitutions will have little effect on binding activity. The great majority of variants will possess at least some cytoprotective or neutralizing activity, particularly if the substitution is conservative. Conservative amino acid substitutions are substitutions from the same class, defined as acidic (Asp, Glu), hydroxy-like (Cys, Ser, The), amides (Asn, Gin), basic (His, Lys, Arg), aliphatic-like (Met, Ile, Leu, Val, Gly, Ala, Pro), and aromatic (Phe, Tyr, Trp).

Homologous antibody or polypeptide sequences generally will be greater than about 30 percent homologous on an identical amino acid basis, ignoring for the purposes of determining homology any insertions or deletions from the selected molecule in relation to its native sequence. The compounds discussed herein, i.e., autoimmune inhibitors for administration to the patient with autoimmune disease and/or for removal, neutralization or inhibition of the autoimmunogen(s) by extracorporeal immunosorption in accordance with the present invention, also include glycosylation variants as well as unglycosylated forms of the agents, fusions of the agents with heterologous polypeptides, and biologically active fragments of the agents, again so long as the variants possess the requisite neutralizing or cytoprotective activity.

In a preferred embodiment of the invention, treatments involving administration of an autoimmune inhibitor to a patient, and treatments involving the extracorporeal exposure of the patient's fluid to an autoimmune inhibitor, may be performed alone or in combination.

Administered autoimmune inhibitor of the invention binds to, neutralizes and/or inhibits the molecule(s) associated with or causing the autoimmune response in the patient. More specifically, administration of the autoimmune inhibitor to a patient results in suppression of pathological humoral and adaptive immunity in the patient. In other words, in accordance with the method of the present invention, treatment of a patient with the autoimmune inhibitor causes the humoral and adaptive immune response of the patient to be inhibited or neutralized over that which was, or would have been, present in the absence of treatment.

A patient is in need of treatment with an autoimmune inhibitor, when the patient is suffering from an autoimmune disease or when the patient has produced autoantibodies.

The autoimmune inhibitor antibody(ies) is also effective when immobilized on a solid support. Examples of such solid supports include, but are not limited to, plastics such as polycarbonate, complex carbohydrates such as agarose and sepharose, and acrylic resins, such as polyacrylamide and latex beads. Techniques for coupling antibodies to such solid supports are well known in the art (Weir et al., “Handbook of experimental Immunology” 4th Ed., Blackwell Scientific Publications, Oxford, England, Chap. 10 (1986); Jacoby et al., Meth. Enzym. 34 Academic Press, N.Y.(1974).

For therapeutic purposes, autoantibody gene products may be generated which include proteins that represent functionally equivalent gene products. For example, an equivalent M3R antibody gene product may contain deletions, including internal deletions, additions, including additions yielding fusion proteins, or substitutions of amino acid residues, but that result in a “silent” change, in that the change produces a functionally equivalent auto-M3R antibody gene product. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Alternatively, where alteration of function is desired, deletion or non-conservative alterations can be engineered to produce altered anti-M3R antibody gene products. Such alterations can, for example, alter one or more of the biological functions of the autoantibody gene product. Further, such alterations can be selected so as to generate autoantibody gene products that are better suited for expression, scale up, etc. in the host cells chosen. For example, cysteine residues can be deleted or substituted with another amino acid residue in order to eliminate disulfide bridges. This applies to any autoimmune M3R molecule and allelic variants thereof, that are identified in an individual.

The autoantibody gene products, peptide fragments thereof and fusion proteins thereof, of the invention can be produced by recombinant DNA technology using techniques well known in the art. Methods that are well known to those skilled in the art can be used to construct expression vectors comprising auto-M3R antibody gene product coding sequences and appropriate transcriptional and translational control signals. For example SEQ ID NO's: 1-2 in Example 1 were used to generate the antibody of the invention. These methods include, for example, in vitro recombinant DNA techniques, synthetic techniques, and in vivo genetic recombination. See, for example, the techniques described in Sambrook, et al., 1989, supra, and Ausubel, et al., 1989, supra. Alternatively, RNA capable of encoding auto-M3R antibody gene product sequences may be chemically synthesized using, for example, synthesizers. See, for example, the techniques described in “Oligonucleotide Synthesis”, 1984, Gait, ed., IRL Press, Oxford.

A variety of host-expression vector systems may be utilized to express the autoantibodies, such as anti-M3R gene products. Such host-expression systems represent vehicles by which the coding sequences of interest may be produced and subsequently purified, but also represent cells that may, when transformed or transfected with the appropriate nucleotide coding sequences, exhibit the anti-M3R gene product of the invention in situ. The efficiency of expression may be enhanced by the inclusion of appropriate transcription enhancer elements, transcription terminators, etc. (see Bittner, et al., 1987, Methods in Enzymol. 153, 516-544).

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, JURKAT, Hep2, VERO, BHK, HeLa, COS, MDCK, 293, 3T3, and W138.

A variety of methods can be employed for the diagnostic and prognostic evaluation of SJS and for the identification of subjects having a predisposition to such autoimmune disorders.

Such methods may, for example, detect the presence of M3R gene mutations, or the detection of either over-, under-, or no expression of M3R protein, or mutants.

Mutations at a number of different genetic loci may lead to phenotypes related to autoimmune disorder, structural and synaptic abnormalities. Ideally, the treatment of patients suffering from such disorders will be designed to target the particular genetic loci comprising the mutation mediating the disorder. Genetic polymorphisms have been linked to differences in drug effectiveness. Thus, identification of alterations in M3R molecules, such as, for example, gene or protein can be utilized to optimize therapeutic drug treatments.

In a preferred embodiment, autoimmune related molecule, such as, for example, M3R, expression levels, mutations, polymorphisms can be detected by using a microassay of for example, M3R nucleic acid sequences immobilized to a substrate or “gene chip” for detection of M3R molecules (see, e.g. Cronin, et al., 1996, Human Mutation 7:244-255). Preferred methods are detailed in the examples which follow.

The level of M3R or any M3R-related receptor molecule gene expression, can also be assayed as described in detail in the examples which follow. Additionally, it is possible to perform M3R gene expression assays “in situ”, i.e., directly upon tissue sections (fixed and/or frozen) of patient tissue obtained from biopsies or resections, such that no nucleic acid purification is necessary. For such in situ procedures (see, for example, Nuovo, G. J., 1992, “PCR In Situ Hybridization: Protocols And Applications”, Raven Press, N.Y.). Standard Northern analysis can be performed to determine the level of mRNA expression of the M3R gene.

To assess the efficacy of cell-based gene therapy, in vitro immunoassays can be used. Antibodies directed against M3R gene products may be used in vitro to determine, for example, the level of M3R antibody gene expression achieved in cells genetically engineered to produce such a gene product. In the case of intracellular gene products, such an assessment is done, preferably, using cell lysates or extracts. Such analysis will allow for a determination of the number of transformed cells necessary to achieve therapeutic efficacy in vivo, as well as optimization of the gene replacement protocol.

The tissue or cell type to be analyzed will generally include those that are known, or suspected, to express the M3R gene. The protein isolation methods employed herein may, for example, be such as those described in Harlow and Lane (1988, “Antibodies: A Laboratory Manual”, Cold Spring Harbor Laboratory Press, Cold Spring Harbor, N.Y.). The isolated cells can be derived from cell culture or from a patient. The analysis of cells taken from culture may be a necessary step in the assessment of cells to be used as part of a cell-based gene therapy technique or, alternatively, to test the effect of compounds on the expression of the M3R gene.

Preferred diagnostic methods for the detection of autoimmune molecules, such as, for example, M3R gene products, conserved variants or peptide fragments thereof, may involve, for example, immunoassays wherein the M3R gene products or conserved variants or peptide fragments are detected by their interaction with an anti-M3R gene product-specific antibody. This can be accomplished, for example, by immunofluorescence techniques employing a fluorescently labeled antibody coupled with light microscopic, flow cytometric, or fluorimetric detection. Such techniques are especially preferred for M3R gene products that are expressed on the cell surface.

M3R-Expressing Cells

In another preferred embodiment, the invention provides a cell induced to express a M3R using an expression vector that contains an isolated nucleic acid encoding the M3R. To produce a cell, expressing a M3R, suitable cell lines, for example, CHO, JURKAT, Hep2, VERO, BHK, HeLa, COS, MDCK, 293, 3T3, and W138, are selected for introduction of an expression vector containing a M3R-encoding nucleic acid. In the examples describe below, the Flp-In CHO cell line (Invitrogen, Carlsbad, Calif.) is used to induce uniform levels of M3R expression in transfected cell lines. For production of a human cell line expressing a M3R antigen, a human cell line such as JURKAT can be used. This cell line is known to express low levels of the M3R protein in its cell membrane, thus ensuring the presence of the machinery necessary for expression of the M3R protein in the appropriate configuration in the cytoplasmic membrane on the cell surface. An exemplary JUKKAT cell line is the Flp-In JURKAT cell line (Invitrogen, Carlsbad Calif.), which provides the above-stated advantages of the Flp-In CHO cells, but in a human cell line. It will be understood, however, that many other combinations of vectors and cell lines are suitable for use in the invention to produce cell lines stably transfected with a M3R antigen. Use of vectors and the like are described, supra.

Antibody Compositions

In one embodiment, anti-M3R antibodies, are administered to a mammal, preferably a human, to reduce the autoimmune response. In another preferred embodiment, anti-M3R antibodies, are administered to a mammal, preferably a human, in combination with other types of treatments (e.g., immunosuppressive agents) to reduce autoimmune response.

Anti-M3R antibodies, variants or fragments thereof can be administered to a mammal, preferably a human, using various delivery systems are known to those of skill in the art. For example, anti-M3R antibodies, variants or fragments thereof can be administered by encapsulation in liposomes, microparticles or microcapsules. See, e.g., U.S. Pat. No. 5,762,904, U.S. Pat. No. 6,004,534, and PCT Publication WO 99/52563. In addition, anti-M3R antibodies, variants or fragments thereof can be administered using recombinant cells capable of expressing the antibodies, or retroviral, other viral vectors or non-viral vectors capable of expressing the antibodies.

Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, 1 gM, IgD, IgA and IgY), class (e.g., IgG₁, IgG₂, IgG₃, IgG₄, IgA₁ and IgA₂) or subclass of immunoglobulin molecule. Examples of immunologically active portions of immunoglobulin molecules include F(ab) and F(ab′)₂ fragments which can be generated by treating the antibody with an enzyme such as pepsin or papain.

An isolated M3R or a fragment thereof, can be used as an immunogen to generate antibodies using standard techniques for polyclonal and monoclonal antibody preparation. The fall-length M3R polypeptide or protein can be used for use as immunogens. An antigenic peptide comprises at least 8 (preferably 10, 15, 20, or 30) amino acid residues of the amino acid sequence of M3R and encompasses an epitope of M3R such that an antibody raised against the peptide forms a specific immune complex with M3R.

In another preferred embodiment, an antibody specific for M3R is used as an immunogen to generate antibodies to detect the presence of such autoantibodies. The generation of anti-M3R autoantibodies is described in detail in the Examples which follow. The detection of anti-M3R antibodies is diagnostic of SJS.

The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction. Alternatively, antibodies specific for a protein or polypeptide of the invention can be selected for (e.g., partially purified) or purified by, e.g., affinity chromatography. For example, a recombinantly expressed and purified (or partially purified) protein of the invention is produced, and covalently or non-covalently coupled to a solid support such as, for example, a chromatography column. The column can then be used to affinity purify antibodies specific for the proteins of the invention from a sample containing antibodies directed against a large number of different epitopes, thereby generating a substantially purified antibody composition, i.e., one that is substantially free of contaminating antibodies. By a substantially purified antibody composition is meant, in this context, that the antibody sample contains at most only 30% (by dry weight) of contaminating antibodies directed against epitopes other than those on the desired M3R protein or polypeptide and/or ant-M3R antibody or fragments thereof, and preferably at most 20%, yet more preferably at most 10%, and most preferably at most 5% and more preferably, at most 0.01% (by dry weight) of the sample is contaminating antibodies. A purified antibody composition means that at least 99.9% of the antibodies in the composition are directed against the desired protein or polypeptide.

In accordance with the invention, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against M3R and/or auto-M3R antibodies can be identified and isolated by screening a recombinant combinatorial immunoglobulin library (e.g., an antibody phage display library) with M3R proteins, polypeptides and fragments thereof. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

In another preferred embodiment, where the antibodies or their fragments are intended for therapeutic purposes, it is desirable to “humanize” them in order to attenuate any immune reaction. Humanized antibodies may be produced, for example by replacing an immunogenic portion of an antibody with a corresponding, but non-immunogenic portion (i.e. chimeric antibodies) (Robinson, R. R. et al., International Patent Publication PCT/U.S.86/02269; Akira, K. et al., European Patent Application 184,187; Taniguchi, M., European Patent Application 171,496; Morrison, S. L. et al., European Patent Application 173,494; Neuberger, M. S. et al., PCT Application WO 86/01533; Cabilly, S. et al., European Patent Application 125,023; Better, M. et al., Science 240:1041-1043 (1988); Liu, A. Y. et al. Proc. Natl. Acad. Sci. USA 84:3439-3443 (1987); Liu, A. Y. et al., J. Immunol. 139:3521-3526 (1987); Sun, L. K. et al., Proc. Natl. Acad. Sci. USA 84:214-218 (1987); Nishimura, Y. et al., Canc. Res. 47:999-1005 (1987); Wood, C. R. et al., Nature 314:446-449 (1985)); Shaw et al., J. Natl. Cancer Inst. 80:1553-1559 (1988); all of which references are incorporated herein by reference). General reviews of “humanized” chimeric antibodies are provided by Morrison, S. L. (Science, 229:1202-1207 (1985)) and by Oi, V. T. et al., BioTechniques 4:214 (1986); which references are incorporated herein by reference).

Suitable “humanized” antibodies can alternatively be produced by CDR or CEA substitution (Jones, P. T. et al., Nature 321:552-525 (1986); Verhoeyan et al., Science 239:1534 (1988); Beidler, C. B. et al., J. Immunol. 141:4053-4060 (1988); all of which references are incorporated herein by reference).

Additionally, recombinant antibodies, such as chimeric and humanized monoclonal antibodies, comprising both human and non-human portions, can be made using standard recombinant DNA techniques. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety). Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Fremont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) BioTechnology 12:899-903).

As described herein, anti-M3R autoantibodies can be used diagnostically to monitor anti-M3R antibody levels within an individual suffering from or susceptible to Sjögren's syndrome as part of a clinical testing procedure. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Further, the antibodies of the invention can be conjugated to a therapeutic moiety and administered to a mammal, preferably a human, to reduce or prevent autoimmune response. Examples of therapeutic moieties that can be conjugated to antibodies to a therapeutic agent Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The antibodies can also be conjugated a drug moiety that modifies a given biological response. For example, a drug moiety may be a protein or polypeptide possessing a desired biological activity. Such proteins may include, for example, a lymphokine such as IL-4 or IL-13.

Kits

The invention also provides kits comprising an M3R-transfected cell and/or antibodies detecting anti-M3R specific autoantibodies conjugated to a detectable substance, and instructions for use and a pharmaceutically acceptable carrier.

Pharmaceutical Compositions

The nucleic acid molecules, polypeptides, antibodies and small molecules (also referred to herein as “active compounds”) described herein can be incorporated into pharmaceutical compositions suitable for administration. Such compositions typically comprise the active compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Such compositions can further include additional active agents.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Intravenous administration is preferred. Solutions or suspensions used for parenteral, intradermal, or subcutaneous application can include the following pyrogen-free components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile, pyrogen-free aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and ftisidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. For antibodies, the preferred dosage is 0.1 mg/kg to 100 mg/kg of body weight (more preferably, 0.1 to 20 mg/kg, 0.1-10 mg/kg, or 0.1 to 1.0 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 0.1 to 1.0 mg/kg, 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a representative, non-limiting example, a subject is treated with one to several (for example, between 3 and 7) doses of an appropriate M3R composition for a maximum of one week. In a preferred embodiment of such an example, treatment would further comprise additional administration approximately once per month for about 3 to 6 months. The preferred route of administration is intravenous bolus injection. It will also be appreciated that the effective dosage of the modulator used for treatment may increase or decrease over the course of a particular treatment. Changes in dosage may result and become apparent from the results of diagnostic assays as described herein. Preferably, administration of modulator is by intravenous injection, and can also be are or near the site of the cells or tissue to be treated, e.g., administration is at or near the site of the autoimmune disorder lesion.

In addition to those compounds described above, the present invention encompasses agents and use of agents which modulate expression or activity of a nucleic acid or polypeptide of interest. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The pharmaceutical compositions can be included in a container, pack, or dispenser together with instructions for administration.

In another embodiment, one or more compositions of the present invention can be administered to a mammal, preferably a human, in combination with one or more standard immunosuppressive or immunomodulatory compounds to reduce or prevent autoimmune response resulting from an autoimmune disorder or an allograft. Examples of immunosuppressive agents include, but are not limited to, azathioprine, corticosteriods (e.g., prednisone), cyclosporine, OKT3 (anti-CD3 monoclonal human antibody), mycophenolate mofetil, rapamycin (sirolimus), mizoribine, deoxyspergualin, macrolide antibiotics such as, for example, FK506 (tacrolimus), brequinar, malononitriloamindes.(e.g., leflunamide), and anti-IL-2R antibodies (e.g., anti-Tac monoclonal antibody and BT 536). See, e.g., Grummet et al., 1999, J. Am. Soc. Nephrol. 10:1366-1388; and Norman and Wadi, eds., 1998, “Primer on Transplantation,” Am. Soc. Tx. Phys, 1st ed.).

Immunosuppressive agents may be administered at high doses initially and then tapered off over time to reduce or prevent autoimmune response. For example, one or more compositions of the invention in combination with an initial dose of cyclosporine ranging from between 5 and 10 mg/kg per day, an initial dose of 10 mg/kg per day prednisone, or an initial dose of 10 mg/kg per day mycophenolate mofetil may be administered to animal to reduce or prevent autoimmune response. Alternatively, one or more compositions of the invention in combination with an initial dose of cyclosporine ranging from between 5 and 10 mg/kg per day, an initial dose of 10 mg/kg per day prednisone, and an initial dose of 10 mg/kg per day mycophenolate mofetil may be administered to animal to reduce or prevent autoimmune response. Preferably, corticosteroids are not administered children.

In another embodiment, one or more compositions of the present invention for modulating the expression or activity of anti-M3R antibody are administered to a mammal, preferably a human, in combination with one or more T cell-targeted or B cell-targeted agents. Examples of such agents include, but are limited to, CTLA-4Ig, IL-2 antagonists (e.g., anti-IL-2 receptor antibodies and IL-2 toxin conjugates), B7 monoclonal antibodies, anti-CD40L monoclonal antibodies, CD4 antagonists (e.g., anti-CD4 monoclonal antibodies), CD3 antagonists (e.g., anti-CD3 monoclonal antibodies), and IL-12 antagonists (e.g., anti-IL-12 monoclonal antibodies and IL-12 toxin conjugates) to reduce or prevent autoimmune response an autoimmune disorder or an allograft.

In order that the invention described herein may be more fully understood, the following examples are set forth. It should be understood that these examples are for illustrative purposes only and are not to be construed as limiting this invention in any manner.

### EXAMPLES

### Example 1

M3R-CHO Cell Lines Expressing Cell Surface M3Rs

This example describes the production of a cell line that expresses the human M3R on the cell surface. The cell line is usefuil for detection of autoantibodies to M3R in immunoassays such as flow cytometry and microscopic assays using immunodetection procedures.

Isolation of human M3R open reading frame. Total mRNA was isolated from the JURKAT human cell line, using oligo-dT columns and converted to cDNA using RT. Amplification of the open-reading frame of human M3R was carried out by RT-PCR using the cDNA as template, in the presence of Taq polymerase and forward and reverse primers as indicated:

| | |
|---|---|
| Forward primer: | |
| CGGAATTCGAGTCACAATGACCTTGCACAA | (SEQ ID NO: 1) |
| Reverse primer: | |
| CAAGGCCTGCTCGGGTGC | (SEQ ID NO: 2) |

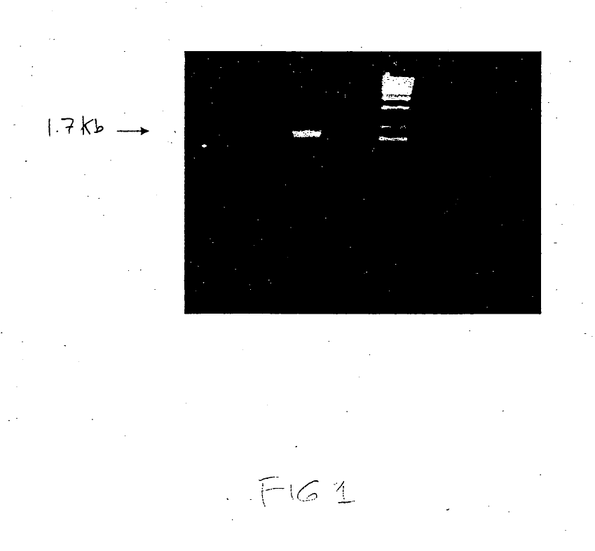

The PCR reaction was initiated with a 5 min incubation at 94° C., followed by 34 cycles of 94° C. for 1 min, 64° C. for 1 min with a step down of 0.3° C. per 6 seconds to 61° C., and 72° C. for 3 min. The reaction was terminated with a 10 min incubation at 72° C. The PCR products were separated by electrophoresis using 0.9% agarose gel and visualized with ethidium bromide. RT-PCR using the above amplimers produced a 1.7 kilobase pair M3R amplicon, as shown in FIG. 1. The PCR product was purified using a gel extraction kit (Qiagen, Valencia, Calif.) and quantified by spectrophotometric measurement of optical density at 260 nm.

Construction of a recombinant vector containing the M3R gene. The PCR products, (containing 3′ A overhangs) were ligated into the cloning vector pcDNA5/FRTNV5-His Topo TA (Invitrogen, Carlsbad, Calif.), which contains 5′ T overhangs, and includes an ampicillin resistance gene and a MCS. Ligation was performed according to the manufacturer's instructions and the resultant recombinant plasmids were used to transform chemically-competent E. coli bacteria.

The E. coli were plated onto LB agar plates containing ampicillin (50 ug/ml) to select for transformed colonies. Plates were incubated overnight at 37° C. Transformed colonies were picked and each selected colony was grown overnight in individual “mini-cultures” containing 3 ml LB broth with 50 ug/ml ampicillin, at 37° C. in a shaking incubator. Plasmid DNA was extracted from each mini-culture using Mini-Preps DNA Purification kit (Qiagen, Valencia, Calif.) following the manufacturer's protocols.

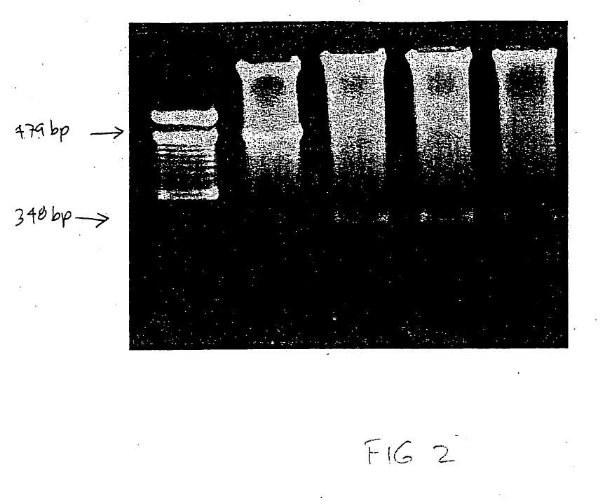

Isolated plasmid DNA was digested using the restriction enzymes Nhe I and Bfr I (Roche Diagnostics, Boehringer Mannheim, Germany) at 37° C. for 2 hours. The digested DNA was size-separated by electrophoresis through 0.9% agarose gel and visualized with ethidium bromide to determine insert orientation. Referring to FIG. 2, after digestion, vectors with inserts in the correct orientation yielded approximately 348 bp and 6519 bp DNA bands, whereas vectors with the opposite directionally inserted DNA yielded 1479 bp and 5388 bp bands, respectively. Bacterial cultures containing vectors with the M3R insert in the proper orientation were grown in LB broth supplemented with 50 ug/ml ampicillin. Selected plasmids were then extracted for further DNA quantitation and sequencing, ensuring fidelity of the M3R gene.

Transfection of Flp-In CHO cells with recombinant pcDNA5/FRT/V5-His Topo TA vectors. Flp-In CHO cells were maintained in UltraCHO medium containing 0.1% zeocin. Transfection of Flp-In CHO cells was carried out with Lipofectin 2000 (Invitrogen, Carlsbad, Calif.). Flp-In CHO cells were treated with a Flp recombinase-expressing plasmid, (i.e., pOG44), and the M3R-expressing pcDNA5/FRTNV5-His vector, at a ratio of 9:1. Flp recombinase was used to mediate insertion of the Flp-In site into the genome at the integrated FRT site through site-specific DNA recombination.

Transfection of the Flp-In CHO cells was carried out by plating 10⁵ cells/well in 6-well microtiter culture plates in 5 ml UltraCHO growth medium (BioWhittaker Cell Biology, Walkersville, Md.) containing fetal bovine serum (FBS) at a concentration of 5%. After a 24 hr incubation, the growth medium was replaced with 0.5 ml serum-free UltraCHO growth medium for 20 min. The cells were then incubated with 9 ug pOG441, 1 ug of M3R plasmid DNA, and 25 ul of Lipofectin 2000. Following a 7 hr incubation at 37° C. and 5% CO₂, the transfection reagents were removed by a single cell wash. The cells were then re-plated in new UltraCHO growth medium with 5% FBS.

Selection of Flp-In CHO cells expressing the recombinant vector. After co-transfection with pOG44 and pcDNA5/FRT/V5-His vector containing the hM3R gene, the Flp-In CHO cells were incubated for 24 hrs to allow for expression of the antibiotic-resistance gene. The growth medium was changed to ProCHO 4 (BioWhittaker Cell Biology, Walkersville, Md.) containing 5% FBS and 0.80 mg/ml hygromycin B (Research Products International Corp. Mt. Prospect, Ill.) for selection of the resistance marker. The cells were supplied with fresh media every 5 days. When necessary, adherent CHO cells were detached from the culture dishes using 0.05% Trypsin-EDTA (Life Technology) and re-plated in new culture dishes. When a stable hygromycin-resistant cell line was established, the cells were transferred from monolayer growth medium to serum-free suspension growth medium (ProCHO 4, BioWhittaker Cell Biology, Walkersville, Md.). The selected cells maintained a hygromycin-resistant, zeocin-sensitive phenotype. No further subdloning was performed.

Confirmation of cell surface expression of M3R. M3R-transfected (M3R-CHO) and control (non-transfected Flp-In CHO) cells, prepared as described above, were collected from growing cultures, washed once with a phosphate-buffered saline (PBS) and resuspended in 100 ul FACS buffer (PBS, 2% AB serum, and 0.01% NaN₃). Approximately 10⁶ cells were incubated for 2 hr at 4° C. with 5 μl of sera from Sj S patients known to have anti-M3R autoantibodies or healthy donors. Cells were then washed with FACS buffer and incubated for 30 min at 4° C. with FITC-conjugated goat anti-human IgG antisera (PharMingen, San Diego, Calif.). After a final wash with FACS buffer, the cells were resuspended in 0.5 ml FACS buffer and analyzed using a FACScan cytometer (Becton Dickinson, Mountain View, Calif.).

Results from the flow cytometric analysis showed a peak of M3R-CHO cells fluorescently labeled on their cell surfaces following incubation with sera from SjS patients, but not from normal subjects, indicating the presence of anti-M3R autoantibodies complexed with M3R antigen expressed on the surfaces of the cells. Control cells (untransfected Flp-In CHO) did not bind antibodies from the SjS patient sera. Similarly, no reaction was observed when either M3R-CHO or control cells were incubated with sera from normal human subjects. These data confirmed that the transfected M3R-CHO cells expressed M3R antigen on their cell surfaces that was effective in forming antigen:antibody complexes with M3R antibodies.

### Example 2

Human Cell Line Expressing M3R

This example describes the production of a human (JURKAT) cell line expressing the human M3R. This cell line is useful for the production of expressed M3R protein, which can be utilized in a predictive diagnostic assay of SjS, such as a radioimmunoassay for detection of autoimmune antibodies to M3R in a patient's serum.

Methods. The cell line Flp-In JURKAT was selected as suitable for transfection with the human M3R because it is known to endogenously express low levels of M3R. Insertion of the human M3R gene into the Flp-In JURKAT cell line was accomplished using procedures described above for Flp-In CHO cells. Transfection of Flp-In JURKAT cells was carried out using recombinant pcDNA5/FRTNV5-His Topo TA vectors expressing the full-length coding sequence of the human M3R gene, amplified from a JURKAT cell line.

### Example 3

Use of M3R Transfected Cells for Diagnosis of SjS

M3R-transfected (M3R-CHO) and control (non-transfected Flp-In CHO) cells were prepared and used for flow cytometric analysis as described in Example 1 above, using sera from normal subjects and subjects with autoimmune diseases. Referring to FIG. 3, typical results are shown from a study in which control and transfected cells were incubated with the sera from subjects with SjS, and from subjects with a connective tissue disease unrelated to SjS. Panels on the left show the graphic readout of the flow cytometer for control cells incubated with sera from the indicated subjects; panels on the right show the readouts for M3R-CHO cells incubated with the corresponding sera. The bar labeled M1 corresponds to the window indicating presence of cells with bound FITC label, i.e., those cells having bound antigen-antibody complexes secondarily labeled with FITC-labeled IgG.

Results showed that no M1 peak was seen with control (non-transfected) cells incubated with any of the tested sera (left panels). In contrast, a sharp M1 peak was observed following incubation of the M3R-CHO cells with sera from the two SjS patients, but not with serum from a non-SjS autoimmune patient (right panels). Similarly, no M1 peak was observed following incubation of the control and M3R-CHO cells with sera from normal subjects. These results demonstrate the usefulness of the M3R-CHO cells for detecting autoantibodies to M3R in the sera of patients with SjS, and for distinguishing between SjS patients and patients with similar diseases of different origin.

Other Embodiments

While the above specification contains many specifics, these should not be construed as limitations on the scope of the invention, but rather as examples of preferred embodiments thereof. Many other variations are possible. Accordingly, the scope of the invention should be determined not by the embodiments illustrated, but by the appended claims and their legal equivalents.

1. A method for diagnosing Sjögren's syndrome in a subject, the method comprising the steps of:
a) obtaining a biological sample from the subject;
b) analyzing the sample of the presence of an antibody that specifically binds a membrane-associated M3R; wherein the presence of the antibody in the sample indicates that the subject has Sjögren's syndrome.

2. The method of claim 1, wherein the biological sample is a fluid selected from the group consisting of blood, blood serum, saliva, tears, mucus and ascites fluid.

3. The method of claim 1, wherein the biological sample is contacted with a cell that expresses a membrane-associated M3R.

4. The method of claim 3, wherein the cell is engineered to express membrane-associated M3R.

5. The method of claim 4, wherein the engineered cell is a JURKAT cell or a CHO cell.

6. The method of claim 1, wherein the biological sample is contacted with a membrane-associated M3R that is not associated with a cell.

7. A method for detecting anti-M3R antibodies in a biological sample, the method comprising the steps of:
(a) obtaining a biological sample;
(b) analyzing the sample of the presence of an antibody that specifically binds a membrane-associated M3R.

8. The method of claim 7, wherein the biological sample is a fluid selected from the group consisting of blood, blood serum, saliva, tears, mucus and ascites fluid.

9. The method of claim 7, wherein the biological sample is contacted with a cell that expresses a membrane-associated M3R.

10. The method of claim 7, wherein the cell is engineered to express membrane-associated M3R.

11. The method of claim 7, wherein the engineered cell is a JURKAT cell, Hep2 cell or a CHO cell.

12. The method of claim 7, wherein the biological sample is contacted with a membrane-associated M3R that is not associated with a cell.

13. A cell expressing a M3R antigen comprising an isolated nucleic acid encoding a M3R antigen.

14. The cell of claim 13, wherein the cell is stably transformed with the nucleic acid encoding a M3R antigen

15. The cell of claim 13, wherein the isolated nucleic acid encodes a human M3R antigen.

16. The cell of claim 13, wherein the M3R antigen is expressed on the cell surface.

17. The cell of claim 13, wherein the cell is a CHO cell, Hep2 cell or a JURKAT cell.


SEQUENCE LISTING (2 sequences)

SEQ ID NO: 1 - DNA, 30 bp, Homo sapiens
  Forward PCR primer (1)..(30)
cggaattcgagtcacaatgaccttgcacaa

SEQ ID NO: 2 - DNA, 18 bp, Homo sapiens
  reverse PCR primer (1)..(18)
caaggcctgctcgggtgc